(19) United States
(12) Patent Application Publication
(10) Pub. No.: US 2013/0018023 A1
(43) Pub. Date: Jan. 17, 2013

(54) COMPOSITIONS FOR TOPICAL ADMINISTRATION

(71) Applicants: Ofer Toledano, Kfar Saba (IL); Karine Neimann, Ness Ziona (IL); Braham Shroot, Princeton, NJ (US)

(72) Inventors: Ofer Toledano, Kfar Saba (IL); Karine Neimann, Ness Ziona (IL); Braham Shroot, Princeton, NJ (US)

(73) Assignee: SOL-GEL TECHNOLOGIES LTD., Ness Ziona (IL)

(21) Appl. No.: 13/637,295

(22) PCT Filed: Mar. 24, 2011
(86) PCT No.: PCT/IL2011/000272

§ 371 Date: Sep. 25, 2012

Related U.S. Application Data
(60) Provisional application No. 61/317,462, filed on Mar. 25, 2010.

(51) Int. Cl. A61K 31/56; A61P 29/00; A61P 17/00; A61K 31/439; A61K 31/351

(52) U.S. Cl. 514/171; 514/304; 514/460

(57) ABSTRACT

The invention provides a composition comprising benzoyl peroxide or derivative thereof; and at least one antibiotic compound selected from pseudomonic acid, retapamulin, fusidic acid and derivatives thereof; methods and uses of a composition of the invention in the treatment of at least one topical disease or disorder and kits comprising a composition of the invention.

### FIELD OF THE INVENTION

This invention relates to compositions and kits comprising benzoyl peroxide or derivatives thereof and at least one antibacterial and/or antimicrobial agent.

### BACKGROUND OF THE INVENTION

As the use of conventional pharmaceutical antibiotics has increased for medical, veterinary and agricultural purposes, there has been a concurrent emergence of antibiotic-resistant strains of pathogenic bacteria.

The emergence of single- or multi-drug resistant bacteria can result from genetic modifications that respond to selective pressures associated with antibiotic use. Over the last several decades, the increasingly frequent usage of antibiotics has acted in concert with spontaneous mutations arising in the bacterial gene pool to produce different strains of bacteria not susceptible to current antibacterial treatments.

Gram-positive bacteria are a major cause of nosocomial infection. The most common pathogenic isolates in hospitals include Enterococcus spp., Staphylococcus aureus, coagulase-negative staphylococci, and Streptococcus pneumoniae (See, e.g., Principles and Practice of Infectious Diseases, 4th ed. Mandell G L, Bennett J E, Dolin R, ed. Churchill Livingstone, New York 1995), many strains of which are resistant to one or more antibiotics.

Vancomycin-resistant enterococcus (VRE) spp. are becoming increasingly common in hospital settings. In the first half of 1999, 25.9% of entercoccal isolates from Intensive Care Units were vancomycin-resistant; an increase from 16.6% in 1996 and from 0.4% in 1989. VRE are also commonly resistant to many other commercial antibiotics, including beta-lactams and aminoglycosides. Thus, patients who are immunocompromised or those having a prolonged hospital stay are at increased risk for acquiring a VRE infection. The problem of antibiotic resistance is not unique to Enterococcus spp. Strains of many other potentially pathogenic Gram-positive bacteria displaying antibiotic resistance have been isolated including methicillin-resistant Staphylococcus aureus (MRSA), vancomycin-resistant Staphylococcus aureus (VRSA), glycopeptide intermediate-susceptible Staphylococcus aureus (GISA), vancomycin-resistant MRSA (VR-MRSA) and penicillin-resistant Streptococcus pneumoniae (PRSP). Like VRE, therapeutic options for treating infections by these organisms are limited.

Resistance transfer is another complicating factor in the management of antibiotic-resistant infections. Vancomycin resistance can transfer from VRE to other Gram-positive bacteria, including S. aureus, in vitro. Thus, the presence of resistant bacteria (e.g., VRE) in a hospital poses not just the risk of infection but also the continued evolution of resistant organisms (e.g., creating more virulent organisms such as VR-MRSA).

A need exists to develop alternative strategies of antibacterial treatment. For example, there exists a need for new compositions and methods of treating or preventing bacterial infection caused by strains of bacteria unsusceptible to current forms of antibacterial treatments.

### SUMMARY OF THE INVENTION

The present invention provides a composition comprising:
a first agent comprising benzoyl peroxide or derivative thereof; and
a second agent comprising at least one antibiotic compound selected from pseudomonic acid, retapamulin, fusidic acid and derivatives thereof.

In another one of its aspects the invention provides a composition consisting of benzoyl peroxide or a derivative thereof and at least one of pseudomonic acid, retapamulin, fusidic acid and derivatives thereof.

Benzoyl peroxide is an organic peroxide consisting of two benzoyl groups joined by a peroxide group, having the following formula:

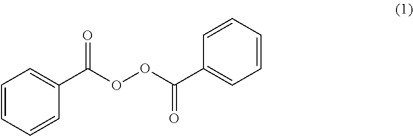 [SMILES: O=C(OOC(=O)c1ccccc1)c1ccccc1]

Derivatives of benzoyl peroxide include but are not limited to compounds of formula (1) being substituted by one to five substitutions on at least one of benzene rings, with a substituent select from C1-C6 alkyl, C2-C6 alkenyl, C1-C6 alkynyl, C1-C 6 cycloalkyl.

The term “antibiotic” agent or compound (used interchangeably with the term “antibacterial” compound or agent), encompasses a compound that can ameliorate or slow down the growth of bacteria, and can also include within its scope antimicrobial, anti-fungal, bacteriocidal agents and compounds.

Pseudomonic acid is a topical antibiotic originally isolated from Pseudomonas fluorescens NCIMB 10586, effective against Gram-positive bacteria, including MRSA. There are several derivatives of pseudomonic acid including pseudomonic acid A, B (with an additional hydroxyl group at C8), C (with a double bond between C10 and C11 instead of the epoxide of pseudomonic acid A), and D (with a double bond at C4′ and C5′ in the 9-hydroxy-nonanoic acid portion), having molecular formulae (2), (3), (4) and (5).

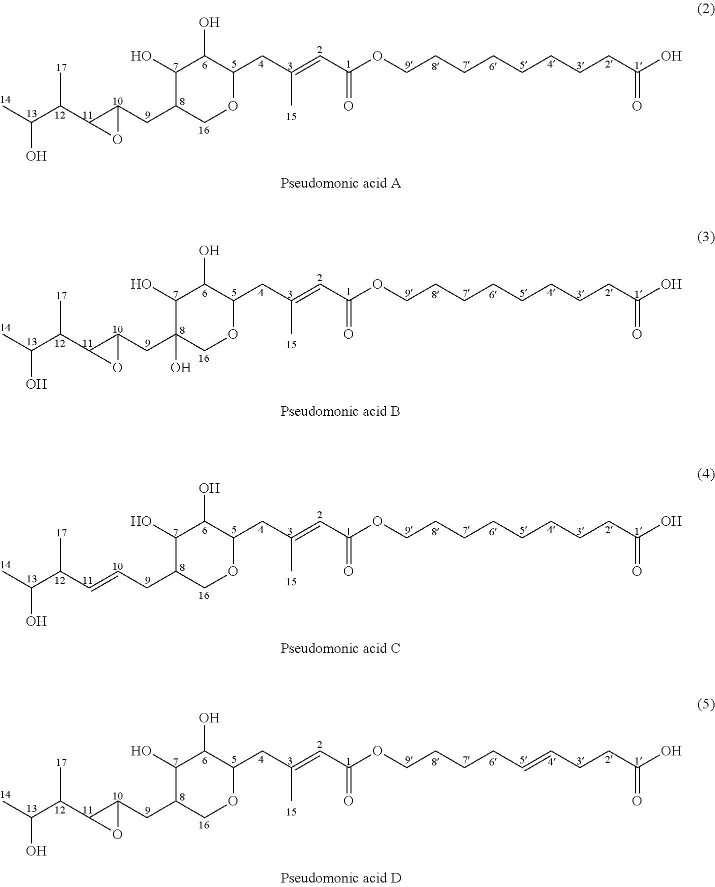 [SMILES: C/C(=C\C(=O)OCCCCCCCCC(=O)O)CC1OCC(CC2OC2C(C)C(C)O)C(O)C1O ; C/C(=C\C(=O)OCCCCCCCCC(=O)O)CC1OCC(O)(CC2OC2C(C)C(C)O)C(O)C1O ; C/C(=C\C(=O)OCCCCCCCCC(=O)O)CC1OCC(C/C=C/C(C)C(C)O)C(O)C1O ; C/C(=C\C(=O)OCCCC/C=C/CCC(=O)O)CC1OCC(CC2OC2C(C)C(C)O)C(O)C1O]

Mupirocin is a mixture of several pseudomonic acids, with pseudomonic acid A constituting greater than 90% of the mixture. Also present in mupirocin are pseudomonic acid B, pseudomonic acid C, and pseudomonic acid D. Mupirocin is bacteriostatic at low concentrations and bactericidal at high concentrations. It is used topically and is effective against Gram-positive bacteria, including MRSA.

Retapamulin is a topical pleuromutilin antibiotic (3aS,4R,5S,6S,8R,9R,9aR,10R)-6-ethenyl-5-hydroxy-4,6,9,10-tetramethyl-1-oxodecahydro-3a,9-propano-3aH-cyclopenta[8]annulen-8-yl{[(1R,3S,5S)-8-methyl-8-azabicyclo[3.2.1]oct-3-yl]sulfanyl}acetate), which inhibits bacterial protein synthesis. Retapamulin utilized by the present invention may be synthetic or semi-synthetic. Derivatives of retapamulin include but are not limited to any pharmaceutically acceptable salt thereof.

Fusidic acid is a bacteriostatic antibiotic (2-[(1S,2S,5R,6S,7S,10S,11S,13S, 14Z,15R,17R)-13-(acetyloxy)-5,17-dihydroxy-2,6,10,11-tetramethyltetracyclo[8.7.0.02,7.011,15]heptadecan-14-ylidene]-6-methylhept-5-enoic, having molecular formula (6)), which inhibits the bacterial protein synthesis by preventing the turnover of elongation factor G (EF-G) from the ribosome. Fusidic acid is effective against gram-positive bacteria such as Staphylococcus species and Corynebacterium species. Fusidic acid utilized by the present invention may be synthetic or semi-synthetic being derived from Fusidium coccineum, Mucor ramannianus or Isaria kogana. Derivatives of fusidic acid include but are not limited to any pharmaceutically acceptable salt thereof (such as for example sodium fusidate).

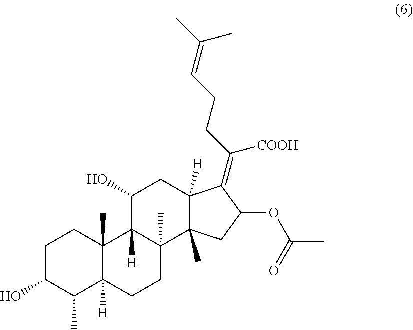

The invention provides a composition comprising a first agent consisting of benzoyl peroxide or a derivative thereof and a second agent comprising a compound selected from pseudomonic acid, retapamulin and derivatives thereof.

The invention further provides a composition comprising a first agent consisting of benzoyl peroxide or a derivative thereof and a second agent comprising a compound selected from retapamulin, fusidic acid and derivatives thereof.

The invention provides a composition comprising benzoyl peroxide or a derivative thereof and at least one pseudomonic acid derivative. The invention provides a composition comprising benzoyl peroxide or a derivative thereof and fusidic acid or a derivative thereof. The invention provides a composition comprising benzoyl peroxide or a derivative thereof and retapamulin or a derivative thereof.

In some embodiments, a composition of the invention comprises benzoyl peroxide in a concentration of at least about 0.5% w/w. In other embodiments, a composition of the invention comprises benzoyl peroxide in a concentration of at least about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5 or 15% w/w. In further embodiments, a composition of the invention comprises benzoyl peroxide in a concentration of between about 0.5 to about 15% w/w. In other embodiments, a composition of the invention comprises benzoyl peroxide in a concentration of between about 0.5-14, 1-10, 2-10, 1-8, 1-5 or 0.5-2% w/w.

In some other embodiments, said second agent comprises at least one pseudomonic acid derivative. In other embodiments, said second agent is mupirocin. In some embodiments, a composition of the invention comprises mupirocin in a concentration of at least about 0.5% w/w. In other embodiments, a composition of the invention comprises mupirocin in a concentration of at least about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5 or 15% w/w. In further embodiments, a composition of the invention comprises mupirocin in a concentration of between about 0.5% to about 5%. In other embodiments, a composition of the invention comprises mupirocin in a concentration of between about 0.5-14, 1-10, 2-10, 1-8, 1-5 or 0.5-2% w/w.

In some other embodiments, a composition of the invention further comprises fusidic acid.

In a further aspect the invention provides a composition comprising a first agent being benzoyl peroxide or a derivative thereof and a second agent selected from pseudomonic acid, retapamulin, fusidic acid or derivatives thereof, for the treatment of a topical condition.

The term “topical condition” as used herein is meant to encompass any condition, disease or disorder manifested on body surfaces such as the skin or mucousal membranes such as the vagina, anus, throat, eyes and ears, including any tissue covering a body of a mammalian subject consisting of the outer, thinner epidermis (epithelial tissue) and the inner, thicker dermis (connective tissue), that is anchored to the subcutaneous layer. It should be noted that a composition or kit of the invention is intended for dermatological use on any type of skin area, being an exterior exposed area (such as for example areas of the skin, scalp, hair, and nails), an interior skin area such as a mucosal membrane (such as for example mucosal membrane around and on the nostrils, the lips, the ears, the genital area, and the anus) or any vicinal areas in close proximity with the treated skin or mucosal membrane areas wherein said composition and agents comprised in said composition may reach via any kind of diffusion mechanisms to a skin area or mucosal membrane.

In a further aspect the invention provides a composition comprising a first agent being benzoyl peroxide or a derivative thereof and a second agent selected from pseudomonic acid, retapamulin, fusidic acid or derivatives thereof, for the treatment of or the prevention of a bacterial infection (e.g., a treatment that inhibits growth of and/or that kills bacteria).

In some embodiments, said bacterial infection is a drug-resistant bacterial infection.

The terms “bacteria” and “bacterium” refer to all prokaryotic organisms, including those within all of the phyla in the Kingdom Procaryotae. It is intended that the term encompass all microorganisms considered to be bacteria including Mycoplasma, Chlamydia, Actinomyces, Streptomyces, and Rickettsia. All forms of bacteria are included within this definition including cocci, bacilli, spirochetes, spheroplasts, protoplasts, etc. Also included within this term are prokaryotic organisms that are Gram-negative or Gram-positive. “Gram-negative” and “Gram-positive” refer to staining patterns with the Gram-staining process, which is well known in the art. (See e.g., Finegold and Martin, Diagnostic Microbiology, 6th Ed., CV Mosby St. Louis, pp. 13-15 (1982)). “Gram-positive bacteria” are bacteria that retain the primary dye used in the Gram stain, causing the stained cells to generally appear dark blue to purple under the microscope. “Gram-negative bacteria” do not retain the primary dye used in the Gram stain, but are stained by the counterstain. Thus, Gram-negative bacteria generally appear red. In some embodiments, bacteria are continuously cultured. In some embodiments, bacteria are uncultured and existing in their natural environment (e.g., at the site of a wound or infection) or obtained from patient tissues (e.g., via a biopsy). Bacteria may exhibit pathological growth or proliferation. Examples of bacteria include, but are not limited to, bacterial cells of a genus of bacteria selected from the group comprising Salmonella, Shigella, Escherichia, Enterobacter, Serratia, Proteus, Yersinia, Citrobacter, Edwardsiella, Providencia, Klebsiella, Hafnia, Ewingella, Kluyvera, Morganella, Planococcus, Stomatococcus, Micrococcus, Staphylococcus, Vibrio, Aeromonas, Plessiomonas, Haemophilus, Actinobacillus, Pasteurella, Mycoplasma, Ureaplasma, Rickettsia, Coxiella, Rochalimaea, Ehrlichia, Streptococcus, Enterococcus, Aerococcus, Gemella, Lactococcus, Leuconostoc, Pedicoccus, Bacillus, Corynebacterium, Arcanobacterium, Actinomyces, Rhodococcus, Listeria, Erysipelothrix, Gardnerella, Neisseria, Campylobacter, Arcobacter, Wolinella, Helicobacter, Achromobacter, Acinetobacter, Agrobacterium, Alcaligenes, Chryseomonas, Comamonas, Eikenella, Flavimonas, Flavobacterium, Moraxella, Oligella, Pseudomonas, Shewanella, Weeksella, Xanthomonas, Bordetella, Franciesella, Brucella, Legionella, Afipia, Bartonella, Calymmatobacterium, Cardiobacterium, Streptobacillus, Spirillum, Peptostreptococcus, Peptococcus, Sarcinia, Coprococcus, Ruminococcus, Propionibacterium, Mobiluncus, Bifidobacterium, Eubacterium, Lactobacillus, Rothia, Clostridium, Bacteroides, Porphyromonas, Prevotella, Fusobacterium, Bilophila, Leptotrichia, Wolinella, Acidaminococcus, Megasphaera, Veilonella, Norcardia, Actinomadura, Norcardiopsis, Streptomyces, Micropolysporas, Thermoactinomycetes, Mycobacterium, Treponema, Borrelia, Leptospira, P. acnes and Chlamydiae.

As used herein, the term “microorganism” refers to any species or type of microorganism, including but not limited to, bacteria, archaea, fungi, protozoans, mycoplasma, and parasitic organisms.

In one of its aspects the invention provides a composition comprising a first agent being benzoyl peroxide or a derivative thereof and a second agent selected from pseudomonic acid, retapamulin, fusidic acid or derivatives thereof, for the treatment of at least one disease or disorder associated with a microorganism or bacteria (pathogene). In some embodiments, said microorganism is selected from the following non-limiting list: Methicillin-resistant Staphylococcus aureus, Coagualse Negative Staphylococcus epidermidis, Vancomycin-Restistant Enterococcus faecali, Escherischia coli, dermatophytes, and Candida albicans.

Such bacterial pathogens can cause a wide variety of diseases and disorders ranging from superficial abscesses (e.g., boils, styes, furuncles and other localized abscesses) to deeper infections (e.g., osteomyelitis, pneumonia, endocarditis, urinary tract infections, septic arthritis, meningitis, post-operative wound infections, septicemia and food poisoning). For example, S. aureus is a major cause of hospital acquired (nosocomial) infection of surgical wounds and S. epidermidis causes infections associated with indwelling medical devices (See, e.g., Silverstein et al., 1990; Patti et al., 1994; Dann et al., 1994).

In another one of its aspects the invention provides a composition comprising a first agent being benzoyl peroxide or a derivative thereof and a second agent selected from pseudomonic acid, retapamulin, fusidic acid or derivatives thereof, for the treatment of at least one topical disease or disorder. Such a topical disease or disorder is selected from primary skin infections (such as for example impetigo contagiosa, erythrasma) and secondary skin infections (such as for example infected wounds, burns, atopic dermatides, acne, psoriasis).

In some embodiments, the topical condition is a disease or disorder selected from acne, infection, inflammation, pruritis, psoriasis, seborrhea, contact dermatitis, skin cancers, skin photoaging, pigmentation, redness, rosacea, and any combination thereof.

In a further aspect the invention provides, a use of a composition comprising a first agent being benzoyl peroxide or a derivative thereof and a second agent selected from pseudomonic acid, retapamulin, fusidic acid or derivatives thereof, for the preparation of a medicament for the treatment of at least one topical disease or disorder. In some embodiments said at least one topical disease or disorder is selected from the primary skin infections and secondary skin infections.

In another aspect the invention encompasses a method of treating a topical condition in a subject in need thereof, said method comprising administering to said subject an effective amount of a composition comprising a first agent being benzoyl peroxide or a derivative thereof and a second agent selected from pseudomonic acid, retapamulin, fusidic acid or derivatives thereof.

The term “treatment” as used herein refers to the administration of a therapeutic amount of a composition of the invention which is effective in one of the following: ameliorating undesired symptoms associated with a disease, disorder, or pathological condition; effective in preventing the manifestation of such symptoms before they occur; effective in slowing down the progression of a disease or disorder; effective in slowing down the deterioration of the disease; effective to prolong the time period onset of remission period; effective in slowing down the irreversible damage caused in a progressive chronic stage of a disorder; effective to delay the onset of said progressive; effective to lessen the severity or cure the disease or disorder; effective to improve survival rates of individuals infected with the disease, or effective to prevent the disease form occurring altogether (for example in an individual generally prone to the disease) or a combination of two or more of the above.

Treatment may further include improving appearance of a topical surface of a subject, encompassing any visible improvement of the condition of a skin area or mucosal membrane treated by a composition or a kit of the invention. The improvement may appear as any change in the skin or mucosal membrane color, smoothness, uniformity, degree, intensity and number of lesions or wounds on the subject's skin or membrane area which may be due to any kind of skin condition or disorder such as in some embodiments bacterial, viral or fungal infection etc.

A composition or formulation of the subject invention may include carriers, adjuvants and emulsifiers, or nontoxic, non-therapeutic, non-immunogenic stabilizers and the like. Effective amounts of such diluent or carrier will be those amounts which are effective to obtain a pharmaceutically acceptable formulation in terms of solubility of components, biological activity, and the like.

In some embodiments, a composition of the invention is formulated for topical administration. Topical formulations include but are not limited to an ointment, a cream, a lotion, an oil, an emulsion, a gel, a paste, a milk, an aerosol, a powder, a foam, a wash, a transdermal patch and any combination thereof.

A topical formulation according to the invention may also comprise a dermatologically, cosmetic or pharmaceutically acceptable carrier, diluent or excipients in which the metal oxide coated active agent particulates may be e.g., dispersed or suspended. The coated active agent may be easily dispersed or suspended in such a carrier, diluent or excipients, by for example mixing to achieve an effective dispersion or suspension. If necessary, high shear forces may be applied to facilitate fast and efficient mixing of the coated particles in the carrier.

In other embodiments, the carrier of a composition of the invention is in the form of a gel, a cream, a lotion, a cleanser, a saturated pad, an ointment, an oil, an emulsion, a paste, a milk, an aerosol, a powder, a foam, a wash etc. In other embodiments, the carrier of a composition of the invention is an oil-in-water cream. In still further embodiments, the dispersing phase is aqueous based and comprises water as a dispersing medium.

It should be noted that a composition of the invention may comprise a further at least one additive, such as but not limited to: a humectant (such as for example propylene glycol, glycerin, butylen glycol or polyethylene glycol), a buffer (such as for example citric acid aqueous solution, ammonium hydroxide solution phosphate buffer, borate buffer or carbonate buffer), a lubricant (such as for example cyclomethicone, dimethycone, castor oil, Iso propyl miristate, caprylic/capric triglyceride or octyl octanoate), an emulsifier (such as for example cetyl alcohol, glyceryl stearate, PEG-75 stearate, Ceteth-20, Steareth-20, Bis-PEG/PPG-16/16 PEG/PPG-16/16 dimethicone, sorbitan mono-oleate or alkyl poly glucoside), a moisturize (such as for example sodium PCA, sodium hyaluronate, panthenol or sodium latate), a soothing agent (such as for example natural herbal extracts such as Anthemis Nobilis flower extract, Chamomilla Recutita, Hamamelis Virginiana, burdock root, Argireline, Arnica Montana Extract, Shea Butter or aloe vera), a perfume, an exfoliating agent (such as for example polyethylene, glycolic acid 70%), a filler, an anti-irritating agent (such as for example allantoin), a chelating agent (such as for example EDTA), a preservative (such as for example imidazolidinyl urea, potassium sorbate, phenoxyethanol, methyl paraben, propyl paraben or benzyl alcohol), a detergent (such as for example polysorbate 20, sodium dodecyl sulfate or ceterimonium chlorid), a coloring agent, an antimicrobial agent (such as for example SD alcohol 40 or Chlorhexidine gluconate), a thickening agent (such as for example xanthan gum, guar gum, carboxy methyl cellulose, Carbomer or ethyl cellulose) and any combinations thereof.

The term “topical administration” as used herein is meant to encompass local administration of a component of a composition or a kit of the invention onto the surface of a skin or mucosal tissue of a subject without inducing any systemic effect.

In this context it should be noted that a composition or a kit of the invention may be administered according to any treatment profile. For example, each agent may be administered concomitantly, sequentially, separately or parallel to the administration of any other agent of a composition or a kit of the invention. Each agent of a composition of the invention may be administered adjacent to any other skin treatment procedure. Each agent of a composition or a kit of the invention may be administered independently either once or a few times a day (for example twice, three to ten times a day).

In some embodiments, the formulations include a controlled-release device or composition where one or several of the components comprised in a composition of the invention are being released in a delayed fashion.

A composition of the invention may be formulated in a solid, semi-solid, or liquid form such as, e.g. suspensions, aerosols, or the like or any other formulation known to a person skilled in the art. In some embodiments, the compositions are administered in unit dosage forms suitable for single administration of precise dosage amounts. The compositions may also include, depending on the formulation desired, pharmaceutically-acceptable carriers as defined above.

In some other embodiments of the present invention, a composition of the invention may be administered in a single dosage form comprising all the components together.

The term “administering” or its other lingual forms as used in the context of the present invention relates to the path by which an agent, a drug, fluid or other substance is brought into contact with the body. The composition is transported from the site of entry to the part of the body where its action is desired to take place. According to one embodiment of the present invention, said administering may be achieved via any medically acceptable means suitable for a composition of the invention or any agent thereof, including rectal, vaginal, nasal, topical, transdermal, or parenteral (including subcutaneous, intramuscular, intrasynovial, intraperitoneal, intradermal and intravenous) administration.

In some embodiments of the present invention compositions may be provided as sustained release or timed release formulations. The carrier or diluent may include any sustained release material known in the art, such as glyceryl monostrearate or glyceryl distearate, alone or mixed with a wax. Micro-encapsulation may also be used. The timed release formulation can provide a composition of immediate and pulsed release throughout the day. The diluent is selected so as not to affect the biological activity of a composition of the invention. Examples of such diluents are distilled water, physiological saline, Ringer's solution, dextrose solution, and Hank's solution.

A composition of the present invention or each component thereof can thus be administered by any means known in the art, such as oral (including buccal and sublingual), rectal, vaginal, nasal, topical, transdermal, or parenteral (including subcutaneous, intramuscular, intravenous, intrasynovial, intraperitoneal and intradermal) administration.

Compositions, methods and kits of the present invention may be used either alone, or in conjunction with other treatment methods known to those of skill in the art for the treatment of a disease or disorder mentioned above. Such methods may include, but are not limited to phototherapy with ultraviolet light, laser therapies, cryotherapy etc. The administration of a composition of the present invention may be conducted before, during or after other such additional therapies. In addition, a composition of the present invention may be administered concurrently with other treatments known to those of skill in the art for the treatment of a disease or disorder mentioned above.

In therapeutic applications, the dosages and administration schedule of components of a composition of the invention may vary depending on the component, the agent, the age, weight, and clinical condition of the recipient patient, and the experience and judgment of the clinician or practitioner administering the therapy, among other factors affecting the selected dosage. An effective amount of a composition is that which provides a medical benefit as noted by the clinician or other qualified observer. The present invention allows for the administration of a composition of the present invention, either prophylactically or therapeutically or in the context of adjuvant or neo-adjuvant treatment.

When provided prophylactically, a composition of the invention may be administered in advance of any symptom. Prophylactic administration of pharmaceutical compositions may serve to prevent or inhibit a topical condition. A composition of the invention may prophylactically be administered to a patient with, for example, a family history of such conditions. Alternatively, administration of a composition of the invention may be administered to a patient exhibiting none-visible symptoms of a topical disease or disorder, however, such diseases or disorders are measurable in none-direct markers (i.e. markers found in the blood of a subject). When provided prophylactically, the dose of a composition of the invention may be reduced to the appropriate prophylactic dosage.

When provided therapeutically, a composition of the invention may be administered at (or after) the onset of a symptom or indication of a topical disease or disorder.

It should be noted that a composition of the invention may be incorporated in any kind of product form such as, but not limited to: a soap including liquid and bar soap, a shampoo, a hair conditioner, a shower gel, including an exfoliating shower gel, a foaming bath product (e.g. gel, soap or lotion), a milk bath, a soapless cleanser, including a gel cleanser, a liquid cleanser and a cleansing bar, moist towelletes, a body lotion, a body spray, mist or gel, bath effervescent tablets (e.g., bubble bath), a hand and nail cream, a bath/shower gel, a shower cream, a depilatory cream, a shaving product e.g. a shaving cream, gel, foam or soap, an after-shave, after-shave moisturizer, a sunscreen lotion, gel or oil, any kind of make-up product in a cream, lotion or cake form, and any combinations thereof, and any other products used for cleansing, post-cleansing, make-up application to the body, including the skin and hair.

In other embodiments, at least one agent of a composition of the invention may be separately administered, simultaneously or sequentially.

The term “administered sequentially” refers to ordered and successive administration of a pharmaceutical composition of the invention or their components comprised in separate containers. Said sequential administration may be 1, 2 or 3 days apart. For example, a topical composition of the invention may be applied once, twice, three times a day or more. The order of addition of the agent of the composition may be optional. Treatment with a composition of the invention may be continued from 2, 3, 4, 5, 6-7 days or more, in accordance with directions given by a qualified care giver.

Thus, the present invention further provides a kit comprising:
a first container comprising at least one agent of a composition of the invention;
a second container comprising at least one agent of a composition of the invention; and instructions for administration of said containers.

For example, instructions may indicate administration of a first container on non-consecutive days and for administration of a second container daily.

In a further embodiment, components of a composition of the invention may be comprised in multiple, i.e. more than two, containers.

In some embodiments, the agents of a composition of the invention in the first and second container are the same. In other embodiments the agents of a composition of the subject invention in the first and second container are different. For ease of storage and administration, compatible agents of a composition of the invention may be placed in one container, separated from other agents of said composition

According to some embodiments of the invention each agent of a composition of the invention, is contained in a separate container. In another embodiment, all agents for administration on a particular day are combined and stored in one container for ease of use and storage. If necessary for stability purposes, the container may be stored frozen and thawed before administration, e.g., by placing in a refrigerator one or two days before administration.

In another aspect the invention provides a kit comprising at least one container comprising a first agent being benzoyl peroxide or derivative thereof and a second agent selected from pseudomonic acid, retapamulin, fusidic acid or derivatives thereof.

The term “container” as used herein refers to any receptacle capable of holding at least one component of a composition of the invention. Such a container may be any jar, vial or box known to a person skilled in the art and may be made of any material suitable for the components contained therein and additionally suitable for short and/or long term storage under any kind of temperature.

In some embodiments, said first and second agents are contained in at least two containers in a kit of the invention. In other embodiments, a kit of the invention is composed of a dual chamber container wherein said first and second agents are contained in each one of the chambers. In other embodiments, a kit of the invention is for use in the treatment of at least one disease or disorder selected from primary skin infection and secondary skin infections.

It should be noted that a composition and/or a kit of the invention may be formed in a manner suitable for any particular condition of a skin area or mucosal membrane to be treated by way of selecting its ingredients, their concentration, the type of formulation and its consistency, the mode and the timing of use based on the condition of the subject, the area of the skin to be treated, the existing of a different treatment regimen, and other factors as known to a medical practitioner.

The term “subject” in the context of the present invention includes human and non-human mammals.

### DETAILED DESCRIPTION OF EMBODIMENTS

In order to understand the invention and to see how it may be carried out in practice, embodiments will now be described, by way of non-limiting example only

### In Vitro Antimicrobial Properties of Compositions Comprising BPO and Mupirocin

### Materials

### Benzoyl Peroxide (BP): Aldrich Cat #517909-100G (Lot Number: 2729711).

### Mupiorocin (MUP): Teva (Lot Number: 83200210108).

### Bovine Calf Serum: Aldrich Cat #-500 mL (Lot Number: 018K8406).

### Vehicle: DMSO (Electron Microspy Science Cat #13390, Lot Number: 090518, CSA #67-68-5).

### Test Organisms

Methicillin-resistant Staphylococcus aureus (ATCC 33592)
Coagualse Negative Staphylococcus epidermidis (Clinical Isolate)
Vancomycin-Restistant Enterococcus faecali (ATCC 115992)
Escherischia coli (ATCC 25922)
Candida albicans (ATCC 10231)

### Storage

Mupirocin: Mupirocin powder was stored at 4 C. A stock solution of 512 μg/mL was created at the beginning of the experiment and stored at 4 C until use.
Benzoyl Peroxide Powdered Benzoyl Peroxide was stored at room temperature. 12% (w/v) Solutions of Benzoyl Peroxide dissolved in DMSO were made fresh immediately prior to each experimental iteration. Solutions were discarded after use. Benzoyl Peroxide was purchased as a 75% BP/25% water mixture. All calculations accounted for the water mixture.
Dimethyl Sulfoxide (DMSO): DMSO was stored at room temperature

### Test Media

Muller Hinton Broth (MHB) was used in this study for all bacteria: BD Cat #211443, Lot numbers 9097650.

Muller Hinton Agar (MHA) was used in this study for all bacteria: BD Cat #211438, Lot number 7155220.
Yeast Dextrose Broth (Y/D B) was used to test C. albicans
Yeast Dextrose Broth contains 10 g/L of yeast extract and 10 g/L of dextrose. Yeast Extract Cat #BD 210933, Lot number 8074851 (Expiration 2010-01-31). Dextrose Cat # BD215530 (Lot Number 6163169).
Yeast Dextrose Agar (Y/D A) was used to test C. albicans
Yeast Dextrose Agar contains 10 g/L of yeast extract and 10 g/L of dextrose and 17 g/L Agar. Agar Cat # BD 214010 (Lot Number 214010).

### Methods

### Preparation of Inoculum:

1. The test organism, (bacteria or yeast) was obtained from frozen cultures.
2. Two days prior to the experiment, a portion of test bacterium from frozen culture collection was streaked and cultured on a non-selective agar plate (e.g. TSA). Plates were then incubated overnight at 37° C.±1° C. and the culture was examined for purity prior to use. All the bacteria colonies demonstrated the same colony morphology and color.
3. The day prior to the experiment, a portion of test organism was inoculated from the above-mentioned plate into MHB and vortexed. Incubate at 37° C.±1° C. for approximately eighteen hours.
4. For Candida albicans (ATCC 10231): The day prior to the experiment a portion of test organism was inoculated from the above-mentioned plate into Yeast/Dextrose Broth and vortexted.
Bacterial MIC determination was conducted according to Kirby-Bauer macrodilution method. The bacterial/yeast density was adjusted to approximately 10⁷ CFU/m; (Approx OD₆₂₅ 0.1±0.05).
Stock solutions of each test article or combination were made by adding each test article to MHB to give an MHB/test article mixture of the following concentrations: Benzoyl Peroxide=6% in MHB and Mupiocin=256 μg/mL in MHB.

### Combination 1:

For bacterial: Mupirocin=256 μg/mL & BP at 0.0469% in MHB
For yeast: Mupirocin=256 μg/mL & BP at 0.0115% in MHB

### Combination 3:

For bacteria: Mupirocin=256 m/mL & BP at 0.0469% in MHB+10% calf serum
For yeast: Mupirocin=256 m/mL & BP at 0.0115% in MHB+10% calf serum

The stock solutions were added to the first tube of the test series. Beginning with the stock solution, serial doubling dilutions were performed through 12 tubes with additional MHA solution. All tests were conducted in triplicate. For comparison a negative control tube (no active with bacteria) and a positive control tube (no active without bacteria) were also prepared. Each tube received sufficient bacterial inoculum to yield a starting population of approximately 10⁵ CFU/mL. The tubes were incubated for approximately 24 h at 37° C.±1° C. and then stored for MIC: The highest dilution (lowest concentration of active) not demonstrating visible outgrowth (turbidity) in three replicates, as assessed by the unaided eye was considered the MIC.

Bacterial MBC determination was conducted according to Kirby-Bauer macro-dilution method. The MBC was determined by sub-culturing an aliquot of medium from each tube that did not demonstrate bacterial outgrowth to an appropriate medium (Muller Hinton Agar). The tubes were incubated for approximately 24 h at 37° C.±1° C. and then scored for their MBC. The highest dilution (lowest concentration of active) not demonstrating visible outgrowth (turbidity) in three replicates, as assessed by the unaided eye was considered the MBC.

### Results

### Mupirocin MIC/MCB

TABLE 1
Mupirocin MIC Summary Data

| | | | Combination 3 |
|---|---|---|---|
| | | Combination 1 | Mup + BP + |
| | Mupirocin | Mup + BP | 10% Calf Serum |
| C. albicans | Greater | Greater | Greater |
| (yeast) | than 256 μg/mL | than 256 μg/mL | than 256 μg/L |
| Staph | 1 μg/mL | Less | Less |
| epidermidis | | than 0.13 μg/mL | than 0.13 μg/mL |
| MRSA | 1 μg/mL | Less | 0.13 μgg/mL |
| | | than 0.13 μg/mL | |
| VRE | 64 μg/mL | 0.5 μg/mL | 1 μg/mL |
| E. Coli | 64 μg/mL | 32 μg/mL | 128 μg/mL |

TABLE 2
Mupirocin MBC& MFC Summary Data

| | | | Combination 3 |
|---|---|---|---|
| | | Combination 1 | Mup + BP + |
| | Mupirocin | Mup + BP | 10% Calf Serum |
| C. albicans | Greater | Greater | Greater |
| (yeast) | than 256 μg/mL | than 256 μg/mL | than 256 μg/L |
| Staph | 32 μg/mL | 32 μg/m/L | 32 μg/m/L |
| epidermidis | | | |
| MRSA | 32 μg/mL | 32 μg/m/L | 32 μg/m/L |
| VRE | Greater | Greater | Greater |
| | than 256 μg/mL | than 256 μg/mL | than 256 μg/mL |
| E. Coli | Greater | Greater | Greater |
| | than 256 μg/mL | than 256 μg/mL | than 256 μg/mL |

When combined with BP, Mupirocin demonstrated an increased potency of MIC against all bacteria tested. It was ineffective against the yeast, C. albicans at the concentrations tested.

The presence of 10% serum reduced the efficacy of the combination of Mupirocin and BP but this decrease was modest when compared with the overall synergy of the two compounds together.

Cidal activity of Mupirocin and Mup+BP was observed against Staph organisms, however the presence of 10% serum did not reduce the potency.

### Benzoyl Peroxide MIC (in DMSO)

For the purpose of comparing the effect of the combination of BPO and Mup, the MIC of BPO alone was tested, as indicated hereinabove. The results are given in Table 3 herein below. As can be seen from the MIC results the concentration of BPO that was found to be effective was higher for most bacterium as compared with the concentration of BPO in the combination.

TABLE 3
BPO in DMSO

| | MIC DATA (DMSO conc.*) |
|---|---|
| C. albicans (yeast) | 0.046% (0.19%) |
| Staph epidermidis | 0.1875% (0.78%) |
| MRSA | 0.1875% (0.78%) |
| VRE | 0.1875% (0.78%) |
| E. Coli | 0.1875% (0.78%) |

*The BP was dissolved in DMSO so these concentrations were included in the data set although the DMSO levels were far below those found to be bacterio/fungostatic for the DMSO alone (12.5%).

1. A composition comprising:
a first agent comprising benzoyl peroxide or derivative thereof; and
a second agent comprising at least one compound selected from pseudomonic acid, retapamulin, fusidic acid and derivatives thereof.

2. A composition according to claim 1, wherein said benzoyl peroxide is in a concentration of at least 0.5% w/w.

3. (canceled)

4. A composition according to claim 1, wherein said second agent is pseudomonic acid or a derivative thereof.

5. A composition according to claim 1, wherein said second agent comprises at least one pseudomonic acid derivative.

6. A composition according to claim 1, wherein said second agent is mupirocin.

7. A composition according to claim 1, wherein said second agent is mupirocin is in a concentration of at least 0.5% w/w.

8. (canceled)

9. A composition according to claim 1, further comprising fusidic acid.

10. A composition according to claim 1, formulated for topical administration.

11-13. (canceled)

14. A method of treating a topical condition in a subject in need thereof, said method comprising administering to said subject an effective amount of a composition comprising: a first agent comprising benzoyl peroxide or derivative thereof; and a second agent comprising at least one compound selected from pseudomonic acid, retapamulin, fusidic acid and derivatives thereof.

15. A method according to claim 9, wherein said topical condition is selected from at least one disease or disorder selected from primary skin infection and secondary skin infections.

16. A kit comprising at least one container comprising a first agent being benzoyl peroxide or derivative thereof and at least one second agent selected from pseudomonic acid, retapamulin, fusidic acid or derivatives thereof.

17. A kit according to claim 10, wherein said first and second agents are contained in at least two containers.

18. A kit according to claim 10, composed of a dual chamber container wherein said first and second agents are contained in each one of the chambers.

19. A kit according to claim 10, for treating at least one topical disease or disorder.
